(19) United States
(12) Patent Application Publication
(10) Pub. No.: US 2013/0267560 A1
(43) Pub. Date: Oct. 10, 2013

(54) SUSTAINED RELEASE PHARMACEUTICAL COMPOSITIONS OF DONEPEZIL

(71) Applicants: Sunilendu Roy, Ahmedabad (IN); Sushrut Krishnaji Kulkarni, Ahmedabad (IN); Ajaykumar Handa, Ahmedabad (IN)

(72) Inventors: Sunilendu Roy, Ahmedabad (IN); Sushrut Krishnaji Kulkarni, Ahmedabad (IN); Ajaykumar Handa, Ahmedabad (IN)

(73) Assignee: CADILA HEALTHCARE LIMITED, AHMEDABAD GUJARAT (IN)

(21) Appl. No.: 13/880,782

(22) PCT Filed: Oct. 24, 2011
(86) PCT No.: PCT/IN11/00738

§ 371 Date: Jun. 20, 2013

(30) Foreign Application Priority Data
Oct. 22, 2010 (IN) 2949/MUM/2010

(51) Int. Cl. A61K 9/00; A61K 31/445

(52) U.S. Cl. CPC A61K 9/0002; A61K 31/445
USPC 514/319

(57) ABSTRACT

The present invention relates to sustained release pharmaceutical compositions of donepezil or pharmaceutically acceptable salts thereof. The compositions of the invention are stable and provide sustained therapeutically effective plasma levels over a twenty four hour period with reduced incidences of cholinergic side effects. The invention also relates to process of making such compositions.

### FIELD OF THE INVENTION

The present invention relates to sustained release pharmaceutical compositions of donepezil or pharmaceutically acceptable salts thereof. The compositions of the invention are stable and provide extended therapeutically effective plasma levels over a twenty four hour period with reduced incidences of cholinergic side effects. The invention also relates to process of making such compositions.

### BACKGROUND OF THE INVENTION

Donepezil is a known reversible inhibitor of acetylcholinesterase. Donepezil and its pharmaceutically acceptable salts have application in the treatment of a variety of disorders, including dementia and attention deficit disorder. In particular, donepezil hydrochloride is employed as a pharmaceutically active agent for the symptomatic treatment of mild to moderate Alzheimer's dementia. Donepezil has the chemical name ((+/−)-2,3-dihydro-5,6-dimethoxy-2-[[1-(phenylmethyl)-4-piperidinyl]methyl]-1H-inden-1-one) and its structural formula is—

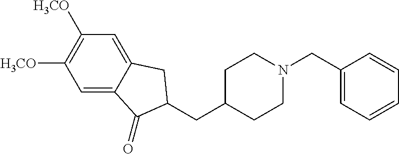 [SMILES: COc1cc2c(cc1OC)C(=O)C(CC1CCN(Cc3ccccc3)CC1)C2]

It is currently formulated as film-coated tablets of 5 mg, 10 mg and 23 mg doses for once a day oral administration under the trade name ARICEPT®.

Donepezil hydrochloride salt has several known crystalline polymorphs having varying levels of stability under conditions of elevated temperature and humidity (as disclosed in U.S. Pat. Nos. 5,985,864, 6,140,321 and 6,245,911).

With the use of an acetylcholinesterase inhibitor, patients may experience cholinergic adverse events when first dosed, especially at higher doses.

Side effects, such as nausea, vomiting, and headaches are more prevalent at initial high doses of acetylcholinesterase inhibitors, such as donepezil, resulting in a reduction of patient compliance. Therefore, an initial therapeutic regimen is often recommended wherein donepezil is first introduced at low doses for several weeks followed by the gradual increase to the active dose for the patient. Formulations that provide sufficient therapeutic amounts of donepezil to the patient while at the same time reducing cholinergic and gastric adverse events would therefore be desirable.

As compared to ordinary rapid-release preparations, a sustained release preparation containing a physiologically active drug allows blood concentrations of the drug to be maintained for a long time at or above the effective therapeutic concentration. Accordingly, by achieving the sustained release characteristics of a drug it is possible to reduce the number of administrations while providing the same or better therapeutic effects, potentially improving compliance, and attenuates adverse events, e.g., related to high plasma peaks. With the sustained release characteristics of the drug, it is also possible to avoid a rapid increase in blood concentration of the drug immediately after administration, thus potentially reducing adverse effects, toxicity and the like due to the drug.

In general, sustained release preparation containing the drug which is physiologically active can be classified into two type preparations; (1) a sustained release coated type preparation, in which release of the drug is controlled by coating a surface of a core particle or a core tablet containing a physiologically active drug with a sustained release coating; (2) a matrix type preparation, in which the drug and a sustained release base are distributed uniformly in the preparation.

Because drug release is affected by the uniformity of the coating in the sustained release coated preparations, the coating conditions of the sustained release coating have to be strictly controlled, and productivity is often low because of long coating times. Further, when applying the sustained release coating to granules, the sustained release coating is generally applied after the drug has been stacked on a core particle generally containing crystalline cellulose or sucrose. Consequently, a size of the preparation tends to be large when multiple layers of the sustained release coating are applied or when a preparation contains a large amount of the drug, making it difficult to administer orally.

On the other hand, the matrix type sustained release preparations have a structure in which the drug and the sustained release base are present uniformly in the preparation and do not require the same strict production conditions as the sustained release coated preparations, and can be produced by manufacturing operations similar to those for the ordinary rapid-release preparations. Accordingly, high productivity can be expected for the matrix type sustained release preparations. In addition, it is easy to prepare the matrix type sustained release preparation even when it contains a large quantity of the drug, and a size of the preparation does not need to be large. Thus, the matrix type sustained release preparations are more useful than the sustained release coated preparations from the standpoint of productivity and smaller size of the preparations.

There are a number of approaches described in prior art to provide sustained release compositions of donepezil.

U.S. Pat. No. 6,372,760 discloses an antidementia medicament composition comprising donepezil which is stabilized by addition of an organic acid selected from group consisting of tosyllic acid, mesyllic acid, benzoic acid, salicylic acid, tartaric acid, citric acid and combinations thereof, wherein the organic acid is not added to form a salt.

PCT Publication No WO 2005/065645 discloses a dosage form comprising amorphous donepezil or an amorphous pharmaceutically acceptable salt thereof and a pharmaceutically acceptable polymeric carrier, wherein the polymeric carrier maintains the active agent in substantially amorphous form.

U.S. Patent Publication No. 2010/0016362 discloses a pharmaceutical composition comprising donepezil or pharmaceutically acceptable salt thereof and a naturally occurring polymer and at least one of edetate, sulfite, dibutylhydroxytoluene and butylhydroxyanisole.

U.S. Patent Publication No. 2009/0208579 discloses a matrix-type sustained release preparation comprising a basic drug or salts thereof which has higher solubility in a 0.1N hydrochloric acid solution and a neutral aqueous solution, pH 6.0 than in basic aqueous solution, pH 8.0; and at least one enteric polymer.

U.S. Patent Publication No. 2008/0248107 discloses an oral controlled release dosage form comprising granules of therapeutically effective amount of active substance having high water solubility and at least one non-polymeric release retardant, and combined with at least one pH-independent non-swelling release retardant, wherein the said dosage form provides controlled release of the active agent with reduced initial burst release.

However, when developing matrix type sustained release preparations of donepezil or a salt thereof using a hydrophobic base, several problems related to dissolution rate and release rate of the drug generally encounter.

Because dissolution rate of donepezil or salt thereof is higher in stomach and lower in intestinal environment, retention time of the drug in stomach may increase, which ultimately, can cause unexpected increase in blood concentration and therefore onset of adverse action may evolve.

On the other side if the formulator attempts to develop a sustained release formulation of donepezil or its salt in order to reduce drug retention time in stomach to prevail the likely adverse effects due to unexpected increase in blood level, by reducing drug release in stomach, the formulation may be excreted with most of the drug remaining in the formulation due to lower release speed of the drug in intestinal environment and thus gives uncertainty about therapeutic efficacy of the formulation administered.

It has also been proved difficult to formulate a sustained release formulation exhibiting release over 24 hour and possessing suitable handling properties, where the drug is one having relatively high solubility, as in the case of donepezil hydrochloride, aqueous solubility of which is pH dependent.

Moreover, donepezil is known to be unstable to light in a formulation with additives, hence attempts have been made previously to stabilize such formulations by using an organic acid. It is also known that in compositions blended with synthetic polymers such as polyvinylpyrrolidone, for example to reduce the bitter taste of the drug, may tend to produce and/or increase level of related substances during storage of such compositions.

Thus, there remains a need in the art to develop a stable sustained release formulation of donepezil or its salt exhibiting release profile suitable for once daily administration which is dependent and/or independent of the pH and also circumvent the cholinergic and gastric side effects alongside rendering optimum absorption of the drug in stomach as well as intestine without initial burst release.

### SUMMARY OF THE INVENTION

In one general aspect, there is provided a sustained release pharmaceutical composition comprising donepezil or salts thereof, one or more pharmaceutically acceptable pH-independent water-swellable rate controlling polymer/s and one or more pharmaceutically acceptable excipients.

In another general aspect, there is provided a sustained release pharmaceutical composition comprising donepezil or salts thereof, one or more pharmaceutically acceptable pH-independent water-swellable rate controlling polymer/s and one or more pharmaceutically acceptable excipients, wherein the ratio of amount of donepezil or its salt to pH-independent polymer/s ranges from 1:10 to 10:1.

In another general aspect, there is provided a sustained release, pharmaceutical composition comprising donepezil or salts thereof, one or more pharmaceutically acceptable pH-independent hydrophilic water-swellable or pH-independent hydrophobic water-swellable rate controlling polymer/s and one or more pharmaceutically acceptable excipients, wherein the ratio of amount of donepezil or its salt to pH-independent polymer/s ranges from 1:10 to 10:1.

In another general aspect, there is provided a sustained release pharmaceutical composition comprising donepezil or salts thereof, one or more pharmaceutically acceptable pH-independent water-swellable rate controlling polymer/s and one or more pharmaceutically acceptable excipients, wherein the amount of pH-independent water-swellable rate controlling polymer/s ranges from about 5% to 80% by weight of the composition.

In another general aspect, there is provided a sustained release pharmaceutical composition comprising donepezil or salts thereof, one or more pharmaceutically acceptable pH-independent water-swellable rate controlling polymer/s and one or more pharmaceutically acceptable excipients, wherein said composition is free of enteric polymer.

In another general aspect, there is provided a sustained release pharmaceutical composition comprising donepezil or salts thereof, one or more pharmaceutically acceptable pH-independent water-swellable rate controlling polymer/s and one or more pharmaceutically acceptable excipients, wherein said composition is free of combination of high molecular weight acidic and basic substances.

In another general aspect, there is provided a matrix-type sustained release pharmaceutical composition comprising donepezil or salts thereof, one or more pharmaceutically acceptable pH-independent water-swellable rate controlling polymer/s and one or more pharmaceutically acceptable excipients.

In another general aspect, there is provided a matrix-type sustained release pharmaceutical composition comprising donepezil or salts thereof, one or more pharmaceutically acceptable pH-independent hydrophilic water-swellable rate controlling polymer/s and pH-independent hydrophobic water-swellable rate controlling polymer/s and one or more pharmaceutically acceptable excipients, wherein the ratio of pH-independent hydrophilic water-swellable rate controlling polymer/s to pH-independent hydrophobic water-swellable rate controlling polymer/s ranges from 15:1 to 1:15.

In another general aspect, there is provided a sustained release pharmaceutical composition comprising multiple unit particles comprising donepezil or salts thereof, one or more pharmaceutically acceptable pH-independent water-swellable rate controlling polymer/s and one or more pharmaceutically acceptable excipients.

In another general aspect, there is provided a sustained release pharmaceutical composition comprising donepezil or salts thereof, one or more pharmaceutically acceptable pH-independent water-swellable rate controlling polymer/s and one or more pharmaceutically acceptable excipients, wherein the composition exhibits no significant difference in both rate and extent of absorption of donepezil or salt thereof as compared to sustained release formulation of donepezil marketed under trade name Aricept® administered once daily.

In another general aspect, there is provided a sustained release pharmaceutical composition comprising donepezil or salts thereof, one or more pharmaceutically acceptable pH-independent water-swellable rate controlling polymer/s and one or more pharmaceutically acceptable excipients, wherein the composition retains at least 80% of the potency of donepezil or salts thereof after storage for 3 months at 40° C./75% RH.

In another general aspect, there is provided a matrix-type sustained release pharmaceutical composition comprising—

0.05% to 5% by weight of donepezil or salts thereof;
about 5% to about 80% by weight of pH-independent water-swellable rate controlling polymer/s; and
further pharmaceutical excipients to 100% by weight.

In another general aspect, there is provided a matrix-type sustained release pharmaceutical composition consisting essentially of—

about 7% to about 13% by weight of donepezil or salts thereof;
about 50% to about 90% by weight of lactose monohydrate;
about 30% to about 60% by weight of corn starch;
about 5% to about 50% by weight of hydroxypropylmethyl cellulose;
about 0.5% to about 5% by weight of polyvinylpyrrolidone;
about 0.05% to about 3% by weight of talc and/or magnesium sterate; and
further pharmaceutical excipients to 100% by weight.

In another general aspect, there is provided a stable sustained release pharmaceutical composition comprising donepezil or salts thereof, one or more pharmaceutically acceptable pH-independent water-swellable rate controlling polymer/s, polyvinylpyrrolidone and one or more pharmaceutically acceptable excipients, wherein the composition is free of antioxidants and/or chelating agents.

In another general aspect, there is provided a sustained release pharmaceutical composition comprising donepezil or salts thereof, one or more pharmaceutically acceptable pH-independent water-swellable rate controlling polymer/s with one or more pharmaceutically acceptable excipients, wherein the composition exhibits a pH-dependent and/or pH-independent release characteristic without initial burst.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect, there is provided a process of manufacturing the sustained release pharmaceutical composition of donepezil or salts thereof comprising—

(a) mixing donepezil or salt thereof with one or more pharmaceutically acceptable pH-independent water-swellable rate controlling polymer/s optionally with one or more pharmaceutically acceptable excipients
(b) granulating or compression molding of above mixture to form granules, pellets, tablets and minitablets.

In another general aspect, there is provided a method of treating a disorder accompanied by acetylcholinesterase activity which comprises administering to a human patient in need thereof a sustained release pharmaceutical composition comprising donepezil or salts thereof, one or more pharmaceutically acceptable pH-independent water-swellable rate controlling polymer/s and one or more pharmaceutically acceptable excipients.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

The details of one or more embodiments of then invention are set forth in the description below. Other features, objects and advantages of the invention will be apparent from the description.

### DETAILED DESCRIPTION OF THE INVENTION

The inventors of the present invention have surprisingly found that it is possible to develop a stable and sustained release formulation of donepezil or its salt which is suitable for once daily administration with reduced cholinergic and gastric side effects without initial burst by employing one or more pH-independent water-swellable rate controlling polymers. It is generally known in the art that use of pH-dependent polymers results in release of the active ingredient, which is dependent on the pH of the environment. However, optimal selection of type and concentration of pH-independent water-swellable polymer have surprisingly found to result in sustained release dosage form which can exhibit pH-dependent and/or pH-independent drug release (in stomach as well as in intestine) without initial burst.

In particular the inventors have found that when one or more pH-independent water-swellable polymers are is used in the judicial weight ratio of 1:10 to 10:1 in matrix-type formulation comprising donepezil or salt thereof, the resulting formulation may exhibit drug release over 24 hour simultaneously reducing cholinergic and gastric adverse events. Such formulations may also remain stable over the storage period.

A sustained release matrix of donepezil or salt thereof may be accomplished by homogeneously embedding drug containing pH-independent water swellable polymer, being a soluble, partially soluble or insoluble network of viscous, hydrophilic polymers, held together by physical or chemical entanglements, by ionic or crystalline interactions, by complex formation, by hydrogen bonds or van der Waals forces. The hydrophilic matrix swells upon contact with water, thereby creating a protective gel layer from which the active ingredient(s) are slowly, gradually, continuously released in time either by diffusion through the polymeric network, by erosion of the gel layer, by dissolution of the polymer, or by a combination of these release mechanisms.

The sustained release composition of donepezil or salt thereof in accordance with present invention was also found to exhibit no significant difference in both rate and extent of absorption of donepezil or salt thereof as compared to sustained release formulation of donepezil marketed under trade name Aricept® administered once daily.

The term “sustained release” as used hereinbefore and throughout the description includes sustained release, controlled release, modified release and delayed release. The term sustained release means release of the active agent at such a rate that blood (e.g., plasma) levels are maintained within a therapeutic range but below toxic levels for at least about 4 hours, preferably at least about 6 hours after administration at steady-state. The term “steady-state” means that a plasma level for a given active agent has been achieved and which is maintained with subsequent doses of the drug at a level which is at or above the minimum effective therapeutic level and is below the minimum toxic plasma level for a given active agent. With regard to dissolution profiles, the first and second dissolution profiles (e.g., in the stomach and in the intestines) should each be equal to or greater than the minimum dissolution required to provide substantially equivalent bioavailability to a capsule, tablet or liquid containing donepezil or salt thereof in an immediate-release form.

The term “donepezil” used throughout the specification refers to not only donepezil per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof. Most preferred salt of donepezil is hydrochloride.

It is possible to use any salts and free base form of donepezil, including polymorphs, hydrates, solvates or amorphous forms.

Suitable “pH-independent water-swellable rate controlling polymers” may include one or more of hydrophilic water-swellable polymers, hydrophobic water-swellable polymers or mixtures thereof.

The pH-independent water-swellable polymer of the invention represents at least one hydrophilic or hydrophobic water-swellable polymer constituting the sustained release matrix which slowly releases the donepezil or its salt. The polymer swells upon contact with aqueous fluid following administration, resulting in a viscous, drug rate controlling gel layer. The viscosity of the polymer preferably ranges from 150 to 100,000 mPas (apparent viscosity of a 2% aqueous solution at 20° C.).

Suitable pH-independent water-swellable rate controlling polymers may include one or more of alkyl celluloses such as methyl cellulose; hydroxyalkyl celluloses, for example, hydroxymethyl cellulose, hydroxyethyl cellulose, hydroxypropyl cellulose, and hydroxybutyl cellulose; hydroxyalkyl alkyl celluloses such as hydroxyethyl methyl cellulose and hydroxypropyl methyl cellulose; carboxyalkyl cellulose esters; ethyl cellulose and its combination with hydroxypropylmethyl cellulose, plasticized ethyl cellulose; microcrystalline cellulose, crosslinked cellulose derivatives such as crosslinked sodium carboxymethyl cellulose; crosslinked polyvinyl pyrrolidone and vinyl acetate (commercially available grade such as Kollidon VA64); other natural, semi-synthetic, or synthetic di-, oligo-, and polysaccharides such as galactomannans, tragacanth, agar, guar gum, and polyfructans; starch and its derivatives such as pregelatinized starch; polyvinyl alcohol; polyvinylpyrrolidone, copolymers of polyvinylpyrrolidone with vinyl acetate; combinations of polyvinyl alcohol and polyvinylpyrrolidone; and polyalkylene oxides such as polyethylene oxide and polypropylene oxide and copolymers of ethylene oxide and propylene oxide, preferably cellulose ether derivatives such as hydroxypropyl methyl cellulose and hydroxypropyl cellulose, and most preferably hydroxypropyl methyl cellulose and the like. Preferred pH-independent water-swellable polymer is hydroxypropyl methyl cellulose.

Depending on the amount of pH-independent water-swellable polymer in the composition, the release profile can be tuned, i.e., larger amounts of pH-independent water-swellable polymers lead to more pronounced sustained release effect and vice versa. Preferably, the amount of pH-independent water-swellable polymers in the composition ranges from 5% to 80% by weight of the composition.

In addition, when using a combination of polymers, the ratio of the polymers also influences the release profile of the composition. A combination of different pH-independent water-swellable polymers can offer possibility of combining different mechanisms by which donepezil is released from the composition. Preferably, the ratio of amount of donepezil or its salt to the amount of pH-independent water-swellable polymer ranges from 10:1 to 1:10.

Different viscosity grades of hydroxypropyl cellulose and hydroxypropyl methyl cellulose are commercially available. Hydroxypropyl methyl cellulose (HPMC) preferably used in the present invention has a viscosity grade ranging from about 3,500 mPas to about 1,00,000 mPas, in particular ranging from about 4,000 mPas to about 20,000 mPas and most in particular a viscosity grade of about 6,500 mPas to about 15,000 mPas (apparent viscosity of a 2% aqueous solution at 20° C.), e.g., hypromellose 2208 or 2206 (DOW, Antwerp, Belgium). HPMC type 2208 contains 19-24% by weight methoxy and 4-12% by weight hydroxypropoxy substituents.

Hydroxypropyl cellulose having a viscosity higher than 1,500 mPas (apparent viscosity of a 1% aqueous solution at 20° C.) is preferred, in particular hydroxypropyl cellulose having a viscosity in the range from about 1500 to about 3000 mPas, preferably from 4000 to 6500 mPas (2% aqueous solutions), e.g., the Klucel series such as Klucel M (Hercules, Wilmington, USA).

According to a preferred embodiment of the matrix of the sustained release composition comprises or essentially consists of hydroxypropyl methyl cellulose, polyvinylpyrrolidone and further excipients. The amount of hydroxypropyl methyl cellulose is preferably in the range from about 1% to about 50%, particularly preferred from about 5% to about 30% by weight of the composition. The amount of polyvinylpyrrolidone is preferably in the range from about 0.5% to about 5%, particularly preferred from about 0.1% to about 3% by weight of the composition. The amount of additional excipients added is up to 100% by weight of the composition.

In a still preferred embodiment, the sustained release composition of donepezil or its salt comprises one or more pH-independent water-swellable rate controlling polymer/s (hydroxypropylmethyl cellulose and polyvinylpyrrolidone), wherein said composition is free of any antioxidant (such as sulfite, ascorbate, dihydroxytoluene and dihydroxyanisole) and chelating agent (such as edetate) and said composition remains stable over the storage period, particularly, the composition retains at least 80% of the potency of donepezil or pharmaceutically acceptable salts thereof in the composition after storage for three months at 40 C and 75% relative humidity.

In a preferred embodiment, the sustained release composition of donepezil or its salt is free of enteric polymer and/or combination of high molecular weight acidic and basic substances.

The term “high molecular weight basic substance” as used herein throughout the specification is a high molecular weight substance which exhibits basic properties when dissolved or suspended in water. For example, in a 2.5% aqueous solution or suspension the high molecular weight basic substance has a pH over 7.0, preferably a pH of 7.5 to 14.0, more preferably 8.0 to 14.0. The term “high molecular weight acidic substance” as used herein throughout the specification is a high molecular weight substance which exhibits acidity when dissolved or suspended in water, for example, with a 2.5% aqueous solution of the high molecular weight acidic substance exhibiting a pH of less than 7.0, preferably a pH of 1.0 to 6.5, more preferably a pH of 1.0 to 6.0.

In a further embodiment, the sustained release composition of donepezil or its salt is pH-dependent and/or pH-independent; preferably release is pH-independent. Therefore, the disadvantage that food related dose-dumping may be encountered is avoided. The problem of food related dose-dumping in fed patients can be attributed to a lot of factors such as the mechanical forces that are exerted by the stomach on its content and thus on an ingested preparation as well as the different pH regions of the gastrointestinal tract. Since the pH values encountered in the gastrointestinal tract vary not only with the region of the tract, but also with the intake of food, an sustained release formulation preferably also has to provide an sustained release profile and in particular has to avoid dose-dumping regardless whether the patient is in fasted or fed conditions.

Accordingly, the present sustained release composition a) retains its pharmacokinetic release profile along its way through the gastrointestinal tract so as to avoid undesirable fluctuations in drug plasma concentrations or complete dose-dumping, in particular avoids dose-dumping in different regions of the gastrointestinal tract.

The sustained release pharmaceutical compositions as described herein may be prepared by processes known to the person having ordinary skill in the art of pharmaceutical technology such as direct compression, wet or dry granulation, slugging, hot melt granulation, hot melt extrusion, fluidized bed granulation, extrusion-spheronization, spray drying and solvent evaporation.

In one embodiment, the sustained release compositions may be prepared by mixing and granulating donepezil or salts thereof with one or more pH-independent water-swellable rate controlling polymers along with one or more pharmaceutically acceptable excipients to form granules. The granules can be dried. The dried granules can be milled, mixed with other pharmaceutically acceptable excipients, lubricated and formulated into suitable dosage form.

In another embodiment, the sustained release composition comprising steps of: mixing and/or granulating donepezil or salt thereof, at least one pH-independent water swellable polymer and optionally other pharmaceutically acceptable excipients, and compression molding the mixture obtained in mixing step.

In still another embodiment, the sustained release compositions may be prepared by mixing and granulating donepezil or salts thereof with one or more pH-independent water-swellable rate controlling polymers along with one or more pharmaceutically acceptable excipients to form granules. The granules can be dried. The dried granules can be milled, mixed with other pharmaceutically acceptable excipients, lubricated and formulated into suitable dosage form. Alternatively, the dosage form can be functionally coated with one or more rate controlling polymers known in the art in order to achieve the desired release pattern.

Suitable dosage form comprises one or more of tablets, multilayered tablets, capsules, pellets, granules, spheroids, beads, minitablets in capsule, pellets in capsule, granules in capsule, powder. Further the powder or granules can be suspended to give a pharmaceutically acceptable oral suspension.

In an embodiment, the sustained release composition is not particularly limited as long as it is an oral preparation. For example, tablets, granules, fine granules, pellets, mini/micro tablets, capsules and the like can be manufactured in the present invention. Capsules can be packed with one or more tablets, granules or fine granules based on the matrix type sustained release preparation according to the present invention. For example, hard capsules can be packed with multiple small-diameter mini-tablets based on the matrix-type sustained release preparation, or with the aforementioned granules or fine granules based on the matrix-type sustained release preparation, or with both tablets based on the matrix sustained release preparation and granules or fine granules based on the matrix-type sustained release preparation. The matrix-type sustained release preparation can also be given a film coating as necessary. It should be noted that the presence or absence of a hydrophilic film coating on the matrix sustained release preparation according to the present invention has very little effect on the dissolution profile of donepezil or salt thereof from the matrix type sustained release preparation.

The sustained release composition of donepezil or its salt is preferably developed into dosage forms such as matrix-tablets/granules/pellets, coated tablets/granules/pellets or multiple unit particles which, alternatively can be filled into capsules or compressed to form tablets.

Alternatively, the sustained release composition of donepezil or its salt can be developed using various osmotic-controlled release oral systems (OROS) known in the art.

The pharmaceutically acceptable excipients may include one or more binders, fillers, lubricants, disintegrants, glidants, colorants, sweeteners, plasticizers and the like.

Suitable fillers may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol and the like.

Suitable disintegrants may include one or more of croscarmellose sodium, sodium starch glycolate, pregelatinized starch, sodium carboxymethyl cellulose, cross-linked polyvinylpyrrolidone and the like.

Suitable binders may include one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins and the like.

Suitable examples of plasticizers include, but not limited to glycerin fatty acid esters; triethyl citrate; propylene glycol; polyethylene glycol and the like.

Suitable lubricants and glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like

Suitable examples of colorants include, but not limited to non-water soluble lake pigments; neutral pigments; yellow ferric oxide; red ferric oxide; black iron oxide and the like.

In a further embodiment, the invention provides a method treating a disease accompanied by acetylcholinesterase activity such as dementia, Alzheimer's disease which comprises administering to a human patient in need thereof a sustained release pharmaceutical composition comprising donepezil or salts thereof and one or more pharmaceutically acceptable pH-independent water-swellable rate controlling polymer/s and one or more pharmaceutically acceptable excipients.

In the context of the present invention, “Bioequivalency” is determined by a 90% Confidence Interval (CI) of between 0.80 and 1.25 for both Cₘₐₓ and AUC under USFDA regulatory guidelines, or a 90% CI for AUC of between 0.80 to 1.25 and a 90% CI for Cmax of between 0.70 to 1.43 under the European regulatory guidelines (EMEA).

The term “confidence interval, (CI)” as used herein refers to the plain meaning known to one of ordinary skill in the art. The confidence interval refers to a statistical range with a specified probability that a given parameter lies within the range.

The term “covariance, (CV)” as used herein refers to the plain meaning known to one of ordinary skill in the art. It is a statistical measure of the variance of two random variables that are observed or measured in the same mean time period. This measure is equal to the product of the deviations of corresponding values of the two variables from their respective means.

The bioequivalence studies were carried out between Aricept® sustained release tablets (reference) and compositions of the invention (test) in fasted and fed states. The study was monitored in terms of Cₘₐₓ and AUC achieved with the test products and the reference product (Aricept®).

The compositions of the invention containing 23 mg of donepezil hydrochloride exhibits pharmacokinetic profile characterized by Cₘₐₓ of about 30.51 to 41.05 μg/ml and AUC₀₋ₜ of about 1050.12 to 1456.48 μg·h/ml.

At 90% confidence interval; area under the concentration time curve (AUCₒ₋ₜ) and maximum plasma concentration (Cₘₐₓ) values of composition of the invention lies between 0.70 and 1.70 as compared to that obtained by a 23 mg sustained release donepezil formulation marketed under the trade name Aricept®.

The relative bioavailability study of donepezil sustained release composition of the invention and donepezil formulation marketed under the trade name Aricept® with varying dose and frequency of administration as demonstrated in Example 5 (Table 9, 10, 11 & 12) concludes that once daily administration of the sustained release formulation explored in present invention provides equivalent extent of absorption compared to once a day donepezil sustained release formulations.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| | | Formula I | Formula II |
|---|---|---|---|
| Sr. No. | Ingredient | (% w/w) | (% w/w) |
| 1 | Donepezil | 10.22 | 10.22 |
| | hydrochloride | | |
| 2 | Lactose monohydrate | 60.67 | 34.44 |
| 3 | Corn starch | 13.33 | 40.00 |
| 4 | Hydroxypropylmethyl | 10.22 | 9.78 |
| | cellulose | | |
| 5 | Polyvinylpyrrolidone | 1.78 | 1.78 |
| 6 | Isopropyl alcohol | q.s. | q.s. |
| 7 | Talc | 0.44 | 0.44 |
| 8 | Magnesium stearate | 1.11 | 1.11 |
| 9 | Opadry brown | 2.22 | 2.22 |
| 10 | Purified water | q.s. | q.s. |
| | Total | 100.00 | 100.00 |

Procedure:

Blend of donepezil hydrochloride containing lactose monohydrate, corn starch and hydroxypropylmethyl cellulose was granulated using binder solution containing isopropyl alcohol and polyvinylpyrrolidone. Granules were then lubricated with talc and magnesium stearate and compressed to form tablets. Resulting tablets were then film coated with Opadry brown dispersion.

### Example 2

TABLE 2

| | | Formula | Formula | Formula | Formula | Formula |
|---|---|---|---|---|---|---|
| Sr. | | I | II | III | IV | V |
| No. | Ingredient | (mg/Tab) | (mg/Tab) | (mg/Tab) | (mg/Tab) | (mg/Tab) |
| 1 | Donepezil | 23.000 | 23.000 | 23.000 | 23.000 | 23.000 |
| | hydrochloride | | | | | |
| 2 | Lactose monohydrate | 144.500 | 149.500 | 159.500 | 164.500 | 169.500 |
| 3 | Hydroxypropylmethyl | 45.000 | 40.000 | 30.000 | 25.000 | 20.000 |
| | cellulose | | | | | |
| 4 | Polyvinylpyrrolidone | 4.000 | 4.000 | 4.000 | 4.000 | 4.000 |
| 5 | Isopropyl alcohol | q.s. | q.s. | q.s. | q.s | q.s. |
| 6 | Talc | 1.000 | 1.000 | 1.000 | 1.000 | 1.000 |
| 7 | Magnesium stearate | 2.500 | 2.500 | 2.500 | 2.500 | 2.5000 |
| 8 | Opadry brown | 5.000 | 5.000 | 5.000 | 5.000 | 5.000 |
| 9 | Purified water | q.s. | q.s. | q.s. | q.s. | q.s. |
| | Total | 225.000 | 225.000 | 225.000 | 225.000 | 225.000 |

Procedure:

Blend of donepezil hydrochloride containing lactose monohydrate and hydroxypropylmethyl cellulose was granulated using binder solution containing isopropyl alcohol and polyvinylpyrrolidone. Granules were then lubricated with talc and magnesium stearate and compressed to form tablets. Resulting tablets were then film coated with Opadry brown dispersion.

TABLE 3

| Time | | Formula | Formula | Formula | Formula | Formula |
|---|---|---|---|---|---|---|
| (hr) | ARICEPT ® | I | II | III | IV | V |
| 0.0 | 0.0 | 0.0 | 0.0 | 0.0 | 0.0 | 0.0 |
| 0.5 | 18.6 | 13.8 | 13.1 | 15.0 | 16.1 | 19.5 |
| 1 | 29.2 | 20.6 | 20.6 | 23.9 | 24.7 | 29.3 |
| 2 | 42.5 | 31.4 | 32.2 | 36.6 | 38.4 | 44.3 |
| 4 | 58.2 | 48.7 | 51.0 | 55.8 | 59.1 | 66.1 |
| 6 | 68.1 | 62.9 | 65.7 | 70.9 | 75.0 | 83.2 |
| 8 | 76.9 | 74.8 | 78.0 | 83.3 | 88.3 | 95.6 |
| 10 | 82.5 | 83.3 | 87.7 | 91.8 | 95.4 | 97.5 |
| 12 | 85.6 | 89.4 | 94.1 | 94.5 | 97.4 | 96.6 |
| 16 | 90.2 | 93.4 | 100.0 | 95.8 | 97.4 | 95.4 |

Table 3 provides dissolution data for donepezil hydrochloride tablet prepared as per Example 2. For determination of drug release rate, USP Type II apparatus (50 rpm) was used wherein 900 mf of water and 0.1N HCl was used as medium.

### Example 3

TABLE 4

| | | Formula | Formula | Formula | Formula |
|---|---|---|---|---|---|
| | | I | II | III | IV |
| Sr. | | (mg/ | (mg/ | (mg/ | (mg/ |
| No. | Ingredient | Tab) | Tab) | Tab) | Tab) |
| 1 | Donepezil | 23.000 | 23.000 | 23.000 | 23.000 |
| | hydrochloride | | | | |
| 2 | Lactose monohydrate | 139.500 | 136.500 | 119.500 | 114.500 |
| 3 | Corn starch | 30.000 | 30.000 | 40.000 | 40.000 |
| 4 | Hydroxypropylmethyl | 20.000 | 23.000 | 30.000 | 35.000 |
| | cellulose | | | | |
| 5 | Polyvinylpyrrolidone | 4.000 | 4.000 | 4.000 | 4.000 |
| 6 | Isopropyl alcohol | q.s. | q.s. | q.s. | q.s. |
| 7 | Talc | 1.000 | 1.000 | 1.000 | 1.000 |
| 8 | Magnesium stearate | 2.500 | 2.500 | 2.500 | 2.500 |
| 9 | Opadry brown | 5.000 | 5.000 | 5.000 | 5.000 |
| 10 | Purified water | q.s. | q.s. | q.s. | q.s. |
| | Total | 225.000 | 225.000 | 225.000 | 225.000 |

Procedure:

Blend of donepezil, hydrochloride containing lactose monohydrate, corn starch and hydroxypropylmethyl cellulose was granulated using binder solution containing isopropyl alcohol and polyvinylpyrrolidone. Granules were then lubricated with talc and magnesium stearate and compressed to form tablets. Resulting tablets were then film coated with Opadry brown dispersion.

TABLE 5

| Time | | Formula | Formula | Formula | Formula |
|---|---|---|---|---|---|
| (hr) | ARICEPT ® | I | II | III | IV |
| 0.0 | 0.0 | 0.0 | 0.0 | 0.0 | 0.0 |
| 0.5 | 18.6 | 16.9 | 17.9 | 13.6 | 13.9 |
| 1 | 29.2 | 27.4 | 28.8 | 22.6 | 22.3 |
| 2 | 42.5 | 44.7 | 45.3 | 35.9 | 35.1 |
| 4 | 58.2 | 71.9 | 69.0 | 56.7 | 54.9 |
| 6 | 68.1 | 94.7 | 87.5 | 73.1 | 70.3 |
| 8 | 76.9 | 103.5 | 100.5 | 85.7 | 81.9 |
| 10 | 82.5 | 103.9 | 102.8 | 96.3 | 90.8 |
| 12 | 85.6 | 103.5 | 103.0 | 99.2 | 96.0 |
| 16 | 90.2 | 104.3 | 103.0 | 98.7 | 98.0 |

Table 5 provides dissolution data for donepezil hydrochloride tablet prepared as per Example 3. For determination of drug release rate, USP Type II apparatus (50 rpm) was used wherein 900 mf of water and 0.1N HCl was used as medium.

TABLE 6

| Time | | Formula | Formula | Formula |
|---|---|---|---|---|
| (hr) | ARICEPT ® | II | III | IV |
| 0.0 | 0.0 | 0.0 | 0.0 | 0.0 |
| 0.5 | 13.9 | 12.9 | 11.4 | 10.4 |
| 1 | 20.0 | 19.9 | 18.2 | 16.6 |
| 2 | 27.8 | 31.7 | 26.7 | 26.1 |
| 4 | 42.0 | 49.7 | 43.6 | 40.6 |
| 6 | 57.3 | 64.7 | 58.4 | 51.8 |
| 8 | 74.5 | 76.8 | 70.3 | 61.7 |
| 10 | 86.1 | 83.9 | 79.1 | 68.8 |
| 12 | 93.3 | 93.1 | 84.5 | 75.1 |
| 16 | 93.3 | 100.0 | 95.1 | 83.7 |

Table 6 provides dissolution data for donepezil hydrochloride tablet prepared as per Formula II, III & IV of Example 3. For determination of drug release rate, USP Type II apparatus (50 rpm) was used wherein 900 mf of water and phosphate buffer of pH 6.8 was used as medium.

### Example 4

### Stability Study

The accelerated stability study of the composition of the invention was conducted at 40° C./75% R.H. over the period of 3 months.

The amount of the impurities measured in the formulation after the storage period indicates that the formulation of the invention is stable under stress conditions.

TABLE 7

| | Related Substances | | | |
|---|---|---|---|---|
| | % known | | | |
| Storage | impurities | Maximum | Total | |
| Period | Related compound B | unknown | Impurity | Assay |
| Initial | BQL | BQL | BQL | 98.7 |
| 1 Month | BQL | BQL | BQL | 100.1 |
| 2 Months | BQL | BQL | BQL | 98.5 |
| 3 Months | BQL | BQL | BQL | 99.9 |

*BQL: Below Quantification Limit

TABLE 8

| | | Formula III | Formula IV |
|---|---|---|---|
| Time (hrs) | ARICEPT ® | (Initial) | (After 3 month) |
| 0.0 | 0.0 | 0.0 | 0.0 |
| 0.5 | 13.9 | 11.4 | 11.3 |
| 1 | 20.0 | 18.2 | 16.9 |
| 2 | 27.8 | 26.7 | 26.3 |
| 4 | 42.0 | 43.6 | 42.3 |
| 6 | 57.3 | 58.4 | 56.0 |
| 8 | 74.5 | 70.3 | 67.2 |
| 10 | 86.1 | 79.1 | 75.9 |
| 12 | 93.3 | 84.5 | 82.8 |
| 16 | 93.3 | 95.1 | 91.7 |
| 20 | 93.8 | 100.0 | 95.6 |
| 24 | 93.7 | 98.6 | 96.3 |

Table 8 provides dissolution profile of donepezil hydrochloride tablet prepared as per Formula III & IV of Example 3 after subjecting to stability study. For determination of drug release rate, USP Type II apparatus (50 rpm) was used wherein 900 mf of water and phosphate buffer of pH 6.8 was used as medium.

The dissolution data of formulations according to Example 2 and 3 indicates that the rate and extent of drug release from the formulation of the present invention is relatively equivalent to that of marketed formulation (Aricept®). The stability data indicates that the composition of the invention remains stable up to 3 months when subjected to accelerated stability conditions.

### Example 5

In-vivo study was conducted in healthy human volunteers to assess bioavailability of donepezil sustained release tablets (23 mg, administered once daily) of the invention with that of Aricept® (Donepezil sustained release tablets) 23 mg (administered once daily).

TABLE 9

| | | Reference | Test |
|---|---|---|---|
| Parameters | Unit | (23 mg OD) | (23 mg OD) |
| Cₘₐₓ | μg/ml | 27.879 | 31.073 |
| AUC₀₋ₜ | H* μg/ml | 1185.951 | 1250.790 |

Table 9 provides summary of Pharmacokinetic parameters of sustained release formulations under Fasting condition.

TABLE 10

| | Ratio | CI_90 | CI_90 |
|---|---|---|---|
| Dependent | [% Ref] | Lower | Upper |
| Ln (Cₘₐₓ) | 111.46 | 105.84 | 117.37 |
| Ln (AUC₀₋ₜ) | 105.46 | 99.72 | 111.55 |

Table 10 provides summary of Pharmacokinetic parameters of sustained release formulations under. Fasting condition.

TABLE 11

| | | Reference | Test |
|---|---|---|---|
| Parameters | Unit | (23 mg OD) | (23 mg OD) |
| Cₘₐₓ | μg/ml | 40.089 | 37.679 |
| AUC₀₋ₜ | H* μg/ml | 1181.242 | 1218.887 |

Table 11 provides summary of Pharmacokinetic parameters of sustained release formulations under Fed condition.

TABLE 12

| | Ratio | CI_90 | CI_90 |
|---|---|---|---|
| Dependent | [% Ref] | Lower | Upper |
| Ln (Cₘₐₓ) | 93.99 | 87.72 | 100.71 |
| Ln (AUC₀₋ₜ) | 103.19 | 99.09 | 107.46 |

Table 12 provides summary of Pharmacokinetic parameters of sustained release formulations under Fed condition.

While the invention has been described in terms of its specific embodiments, certain modifications and equivalents will be apparent to those skilled in the art and are intended to be included within the scope of the invention.

We claim:

1. A sustained release pharmaceutical composition comprising donepezil or salts thereof, one or more pharmaceutically acceptable pH-independent water-swellable rate controlling polymer/s and one or more pharmaceutically acceptable excipients.

2. The sustained release pharmaceutical composition as claimed in claim 1, wherein the pH-independent water-swellable rate controlling polymer/s comprises pharmaceutically acceptable pH-independent hydrophilic water-swellable polymer, pH-independent hydrophobic water-swellable rate controlling polymer/s, or mixture thereof.

3. The sustained release pharmaceutical composition as claimed in claim 1, wherein the pH-independent water-swellable rate controlling polymer/s comprises methyl cellulose, hydroxymethyl cellulose, hydroxyethyl cellulose, hydroxypropyl cellulose, hydroxybutyl cellulose, hydroxyethyl methyl cellulose, hydroxypropyl methyl cellulose, ethyl cellulose, plasticized ethyl cellulose, microcrystalline cellulose, crosslinked sodium carboxymethyl cellulose, crosslinked polyvinyl pyrrolidone and vinyl acetate, galactomannans, tragacanth, agar, guar gum, starch, pregelatinized starch, and copolymers of ethylene oxide and propylene oxide.

4. The sustained release pharmaceutical composition as claimed in claim 1, wherein the pH-independent water-swellable rate controlling polymer is hydroxypropyl methyl cellulose having viscosity ranging from about 3,500 mPas to about 1,00,000 mPas.

5. The sustained release pharmaceutical composition as claimed in claim 1, wherein the pH-independent water-swellable rate controlling polymer is hydroxypropyl cellulose having viscosity ranging from about 1,500 mPas to about 3,000 mPas.

6. The sustained release pharmaceutical composition as claimed in claim 1, wherein the composition comprises about 5% to about 80% by weight of pH-independent water-swellable rate controlling polymer/s.

7. The sustained release pharmaceutical composition as claimed in claim 1, wherein the composition comprises donepezil or its salt and pH-independent polymer/s in weight ratio ranging from 1:10 to 10:1.

8. The sustained release pharmaceutical composition as claimed in claim 1, wherein the composition is free of enteric polymer.

9. The sustained release pharmaceutical composition as claimed in claim 1, wherein the composition is free of high molecular weight acidic and/or basic substances.

10. The sustained release pharmaceutical composition as claimed in claim 1, wherein the compositions is free of antioxidants and/or chelating agents.

11. The sustained release pharmaceutical composition as claimed in claim 1, wherein the composition further comprises polyvinylpyrrolidone.

12. The sustained release pharmaceutical composition as claimed in claim 11, wherein the composition comprises about 0.5% to about 5% by weight of polyvinylpyrrolidone.

13. The sustained release pharmaceutical composition as claimed in claim 11, wherein the compositions is free of antioxidants and/or chelating agents.

14. The sustained release pharmaceutical composition as claimed in claim 1, wherein the composition exhibits a pH-dependent and/or pH-independent release characteristic without initial burst.

15. The sustained release pharmaceutical composition as claimed in claim 1, wherein the composition comprises matrix of donepezil or salts thereof and one or more pharmaceutically acceptable pH-independent water-swellable rate controlling polymer/s.

16. The sustained release pharmaceutical composition as claimed in claim 1, wherein the composition is in the form of multiple unit particles.

17. The sustained release pharmaceutical composition as claimed in claim 1, wherein the composition exhibits no significant difference in both rate and extent of absorption of donepezil or salt thereof as compared to extended release formulation of donepezil marketed under trade name Aricept® administered once daily.

18. The sustained release pharmaceutical composition as claimed in claim 1, wherein the composition retains at least 80% of the potency of donepezil or salts thereof after storage for 3 months at 40° C./75% RH.

19. A matrix-type sustained release pharmaceutical composition comprising—
0.05% to 5% by weight of donepezil or salts thereof;
about 5 to about 80% by weight of pH-independent water-swellable rate controlling polymer/s; and
further pharmaceutical excipients to 100% by weight.

20. A matrix-type sustained release pharmaceutical composition consisting essentially of—
about 7% to about 13% by weight of donepezil or salts thereof;
about 50% to about 90% by weight of lactose monohydrate;
about 30% to about 60% by weight of corn starch;
about 5% to about 50% by weight of hydroxypropylmethyl cellulose;
about 0.5% to about 5% by weight of polyvinylpyrrolidone;
about 0.05% to about 3% by weight of talc and/or magnesium sterate; and
further pharmaceutical excipients to 100% by weight.

21. A process of manufacturing the sustained release pharmaceutical composition as claimed in claim 1 comprising—
(a) mixing donepezil or salt thereof with one or more pharmaceutically acceptable pH-independent water-swellable rate controlling polymer/s optionally with one or more pharmaceutically acceptable excipients
(b) granulating or compression molding of above mixture to form granules, pellets, tablets and minitablets.

22. A method of treating a disorder accompanied by acetylcholinesterase activity comprising administering to a human patient in need thereof a sustained release pharmaceutical composition comprising donepezil or salts thereof, one or more pharmaceutically acceptable pH-independent water-swellable rate controlling polymer/s and one or more pharmaceutically acceptable excipients.
